(19) United States
(12) Patent Application Publication
(10) Pub. No.: US 2005/0154320 A1
(43) Pub. Date: Jul. 14, 2005

(54) Methods and devices for accurate pressure monitoring

(76) Inventors: Michael A. Froelich, Gainesville, FL (US); Richard J. Melker, Gainesville, FL (US); Johannes H. van Oostrom, Gainesville, FL (US)

Correspondence Address: BEUSSE BROWNLEE WOLTER MORA & MAIRE, 390 N. ORANGE AVENUE, SUITE 2500, ORLANDO, FL, 32801, US

(21) Appl. No.: 10/755,071

(22) Filed: Jan. 9, 2004

(51) Int. Cl. A61B 5/02; A61B 5/00

(52) U.S. Cl. 600/486; 600/561; 5/608

(57) ABSTRACT

Disclosed herein are methods, devices and systems for accurately monitoring various pressures of a patient. The invention embodiments herein allow for taking into account pressure changes attributed to changes in elevation and tilt attitudes of the patient. Further disclosed herein is a unique and new pressure correction unit that comprises a tilt sensor affixed thereto or integrated therein.

### FIELD OF THE INVENTION

The subject invention relates to the field of medicine broadly, and specifically to the field of measuring pressure values of a patient during medical procedures.

### BACKGROUND

During certain invasive medical procedures, it is important that intravascular pressures of the patient be accurately measured and monitored. Central Venous Pressure (CVP) is often used to assess the hydration status of a patient. Physicians often refer to the “volume status” of a patient. Conventional measurement of the CVP involves a catheter that is inserted into the vena cava and connected to a pressure transducer. A pressure transducer measures the difference of the CVP between the tip of the catheter (1) and the atmospheric pressure, measured through an opening at the level of the transducer unit (2). The pressure at the tip of the catheter equals the sum of the atmospheric pressure (pₐₜₘ) and the fluid pressure (CVP) in the superior vena cava. The pressure at the transducer unit equals the atmospheric pressure (pₐₜₘ). When calculating differential pressure by subtracting the pressure obtained at the transducer unit from the pressure obtained at the catheter tip, the atmospheric pressure cancels out and the number obtained reflects just the fluid pressure (differential pressure=(pₐₜₘ+CVP)−pₐₜₘ=CVP.)

This principle only works if the transducer unit is exactly at the same level as the tip of the catheter. If this condition is not met a third pressure, the hydrostatic pressure of a fluid filled column will change the presumed CVP. The different measurement errors are illustrated in FIG. 1. Measurement errors can result from elevation or lowering of the patient if the transducer unit is at a fixed height (e.g. attached to an IV-pole, B and C) or tilting of the bed around the transverse (D and E) or Longitudinal axis (F and G).

During many surgical procedures, and sometimes during treatment of intensive care patients, the patients must be elevated and/or tilted. When a CVP line is present, the pressure transducer must be realigned with the right atrium of the heart after every change in position of the patient. This can be a cumbersome and time-consuming procedure, especially in cases where the patient is repositioned several times. In most cases, the physician or nurse relies on the best estimate of alignment by “eyeballing” where the pressure transducer should be placed. Sometimes a carpenter's level is used to assist in alignment. Furthermore, the realignment of the pressure transducer raises the risk of encroaching and contaminating the “sterile field” of the surgical procedure.

As illustrated above, the overlaying hydrostatic pressure will alter CVP measurements if the catheter tip is not perfectly aligned with the transducer unit. Also, another low-pressure parameter, the pulmonary artery wedge pressure (PAWP) is affected in a similar fashion. The PAWP can be measured using a special catheter, the Swan-Ganz catheter.

There have been some attempts to address the problems associated with realigning pressure transducers during surgery. For example, the Delta Press Indicator developed by Weiss, The Internet Journal of Anesthesiology, 1999, Vol. 3, No. 2., utilizes a reference sensor that is attached to the patient's lateral thoracic wall at heart level. While this system is a good intentioned attempt to address certain problems, it falls short in several ways. First the addition of a reference sensor that is attached to the patient's thoracic wall adds yet an additional tube in the field of surgery. Thus, while the Delta Press Indicator attempts to address one problem it creates an additional problem of increasing “bed clutter” and potentially contaminating the sterile field. In addition, the Delta Press Indicator does not account for the significant changes in elevation of the right atrium caused by tilting the patient, or worse can create inaccurate readings when the patient is tilted. In the example of tilting the patient to the left, simple trigonometry dictates that the elevation of the heart will not equal the elevation of the right thoracic wall of the patient.

Accordingly, it is desirable to have a system that overcomes the drawbacks of current methods of obtaining pressure measurements that also accounts for the multiple changes in tilt and elevation that occur during many medical procedures. It is desirable that such system would not increase bed clutter or increase the risk of contaminating the sterile window.

### SUMMARY OF THE INVENTION

The subject invention relates to a novel device and system for achieving precise invasive pressure monitoring that takes into account the change in positioning of the patient that occurs during many medical procedures. As illustrated in FIG. 1, corrections are typically necessary for three types of movements, elevation and lowering of the patient (B and C), rotation around the transverse axis (D and E) and rotation around the longitudinal axis (F and G).

In a specific embodiment, the subject invention relates to a “pressure correction system”. This system preferably consists of four elements; first, a standard detector (e.g. catheter) connected to a transducer (transducer 1); second, a reference detector, preferably positioned under the patient and in vertical alignment (under the patient's heart) with the tip of the first detector, wherein the reference detector is connected to a second pressure transducer mounted to a stable reference point (for example the IV pole, transducer 2). The reference sensor is used to correct for changes in table height. Third, a tilt meter (inclinometer) for the transverse axis and fourth, a tilt meter for the longitudinal axis. The latter three elements provide pressure measurements that will be used to calculate the difference of the height of the catheter tip and the first transducer unit. Alternatively, elements 3 and 4 may be combined into one inclinometer or accelerometer, or similar device that measures or senses tilt in 2 or more axes. The height or angular changes result in changes of hydrostatic fluid pressure that are registered at the second pressure transducer. Tilt changes also result in relative changes of the catheter height in relationship to the first transducer unit. Those changes will be calculated using trigonometry based upon the measured tilt angles and the predetermined spatial relationship of the “pressure correction unit” (FIG. 3).

The subject invention provides an accurate, practical and inexpensive solution for compensating for changes in tilt and height of a patient in hemodynamic monitoring. The subject invention avoids the need for overlaying various hydrostatic components over a patient during invasive procedures. Furthermore, the subject invention avoids the potential errors and difficulties associated with conventional methods of ‘eyeballing’ a midaxillary line to generate a reference measurement. The subject invention also accounts for tilting of the patient, which can be difficult to do during many invasive procedures.

These and other advantageous aspects of the subject invention are described in further detail below.

### DESCRIPTION OF THE DRAWINGS

FIG. 1 shows a series of different changes in patient position that can create CVP measurement errors. FIG. 1A represents the normal patient position. FIG. 1B shows an elevated position which creates an erroneously higher than actual CVP reading. FIG. 1C shows a lowered position which creates an erroneously lower than actual CVP reading. FIG. 1D shows a Trendelenburg Position which creates an erroneously higher than actual CVP reading. FIG. 1E shows a reverse Trendelenburg Position which creates an erroneously lower than actual CVP reading. FIG. 1F shows a right tilt position which creates an erroneously higher than actual CVP reading. FIG. 1G shows a left tilt position which creates an erroneously lower than actual CVP reading.

FIG. 2 shows a typical arrangement of an embodiment of the subject system comprising a first catheter in the patient, a first transducer positioned on an IV pole, a reference catheter strategically positioned under the patient such that it aligns with the first catheter, and a second transducer.

FIG. 3 is a diagram depicting a method of calculating trigonometrically changes in height of a catheter in a patient as a function of tilt.

FIG. 4A is a side view of an embodiment of a reference pressure correction unit embodiment which may be used in accord with the teachings herein.

FIG. 4B is a top view of the embodiment shown in FIG. 4A showing examples of inclinometer that may be used in conjunction with the reference pressure correction unit.

FIG. 5 shows a top view of a patient transparent to illustrate the respective placement of the reference pressure correction unit as shown in FIG. 2.

FIG. 6 shows a side view of a bracket embodiment useful in aligning the patient and reference pressure correction unit according to the teachings herein.

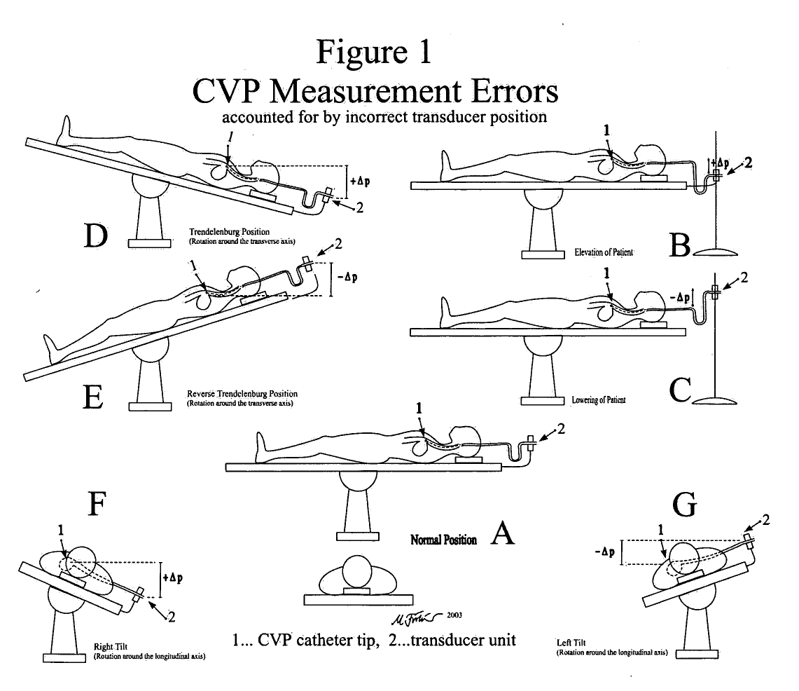

### DESCRIPTION OF THE PREFERRED EMBODIMENTS

The inventors of the present invention have observed that invasive pressure monitoring, e.g. CVP monitoring, is critically and dramatically affected by the relative positioning of the patient and the pressure transducer. For example, if a patient is moved up or down or tilted left or right in relationship to the pressure transducer, the pressure measurement will be changed by the difference between patient and transducer. A patient with a CVP of 5 mm Hg (Torr) is elevated 3 cm per surgeons request, and the pressure transducer is not moved with the patient, the CVP readout will be increased by that pressure difference to now read 5+3 or 8 mmHg (Torr). Conventionally, in order to avoid incorrect readings, the pressure transducer is repositioned and set whenever the patient's position is changed. This has been traditionally accomplished using crude techniques such as a carpenter's level, a laser leveling device, or even by “eyeballing” the reference point. Critical Care Nurse, Volume 20, No. 6 (2000). Obviously, such techniques are prone to human errors, and as noted above, slight changes can significantly affect the measurement. “Invasive hemodynamic monitoring: concepts and practical approaches.” Ann Med. 1997;29:313-318; and “Frequency requirements for zeroing transducers in hemodynamic monitoring”, Am J Crit Care, 1995, 4:465-471.

The subject invention provides an easy and accurate system and methods to account for the repositioning and tilting of a patient when conducting invasive pressure monitoring of the patient. The subject system and methods avoid the need for repeated zeroing and re-referencing of the pressure transducer(s). The subject invention provides a reference sensor that is aligned with a patient's heart. The sensor is positioned such that any changes in elevation of the patient will affect the patient sensor and reference sensor equally. Thus, any change in pressure of the patient brought about by changes in the elevation of the patient can be offset by the pressure reading of the reference sensor. This could be accomplished with a simple algorithm and using a basic microcontroller, possessing basic programming.

Furthermore, tilting of the patient can also increase or decrease the elevation of the pressure detector in the patient (e.g. catheter tip at atrium of heart for CVP measurement). To account for changes in elevation caused by tilting of the patient, the a tilt sensor is implemented for use in conjunction with the reference sensor. Note that the terms “tilt sensor” and “inclinometer” are used interchangeably herein and refer to any device that can detect tilt and generate electrical signals corresponding to such tilting. As per the subject invention, the tilt sensor provides electrical signals representing changes in the tilting of the patient. These electrical signals are sent to a microcontroller (or similar hardware/software combination) possessing appropriate programming to interpret the tilt and calculate changes in elevation brought about by the tilting of the patient. This calculation can then be factored into the calculation of the pressure measurement of the patient. The microcontrollers used for calculating changes in elevation and tilt may be separate or a unified controller. Furthermore, either of the microcontrollers or both may be integrated with other electronic equipment such as a vital signs monitor, or other equipment used to measure and generate invasive pressure readings. Currently used pressure transducers systems consist of disposable transducers and electronic signal conversion modules as manufactured by Abbott®, Baxter/Edwards®, Hewlett-Packard® and Spectramed®.

Those skilled in the art will appreciate that numerous types of tilt sensors may be used in accord with the teachings herein. The type of tilt sensor is not critical so long as it can sense tilt on at least one axis. Preferably, the tilt sensor senses tilt in two axes; this will allow tilt to be sensed and calculated at any angle in a 360 degree circumferential plane. Examples of tilt sensors include, but are not limited to, micromachined accelerometers (Analog Devices, Inc.), electrolytic fluid tilt sensors, mercury switches, ball and tube switches, and Reed switches, and the like.

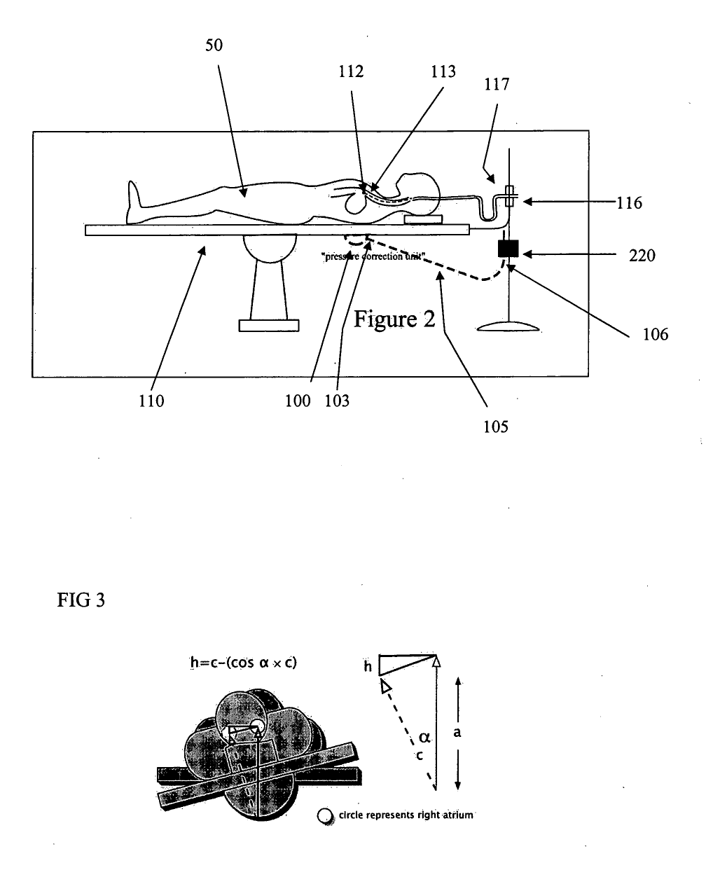

In a preferred embodiment, the tilt sensor comprises a micromachined accelerometer that detects tilt in two axes, such as the ADXL-202 (Analog Devices, Inc.). Those skilled in the art will appreciate that the necessary wiring and circuitry may be provided and implemented in conjunction with the tilt sensor in order that it perform the necessary tilt sensing function, and relay the required electrical information to a pressure monitoring system to calculate the tilt. The tilt sensor could be provided with the necessary electrical circuitry, microcontroller and software to determine the appropriate values, which may then be manually inputted into the pressure monitoring system, or they may be automatically communicated to a pressure monitoring system. Naturally, the pressure monitoring system could be configured with the necessary means to receive the raw values from the tilt sensor to then calculate tilt degree and directions (according to the trigonometric principles displayed in FIG. 3).

Further, those skilled in the art will appreciate that anatomical studies may be conducted to generate a table of standards based on height and weight of the patient, or may be manually measured. In other words, standardized values for changes in elevation correlating to a degree and direction of tilt of a patient may be established. When a tilt degree and direction value is generated, that information may be used by the appropriate medical personnel to calculate the corresponding elevation difference based on the standardized value (using weight and height as parameters), which is then manually entered into a pressure monitoring system. This would avoid the necessity for a tilt sensor, though less preferred.

Preferably, the pressure monitoring system comprises means to automatically receive the tilt degree and direction values, and calculate the elevational difference correlating to the specific tilt degree and direction of the patient. The elevation difference value, as described above, may then be used to accurately reach a pressure value that accounts for the tilting of the patient.

Turning to the figures, in FIG. 2 there is shown a patient 50 lying on an operating table 110. At the opening of the right atrium of the patient's heart 112 has been placed a distal end 113 of a catheter 114 for detecting CVP. The catheter 114 is connected at its proximal end 117 to a first transducer 116. Under the operating table 110 is placed a pressure correction unit 100. The pressure correction unit 100 comprises the distal end 103 of a line 105 (preferably a catheter) which is attached to or integrated with a casing or bracket 115. The line 105 communicates pressure information from the distal end 103 to a pressure transducer 220 connected to the proximal end 106 of the line 105. Preferably, the line 105 is a fluid-filled or air filled catheter whereby the raising or lowering of the distal end causes an increase or decrease in pressure that is processed by the transducer 220.

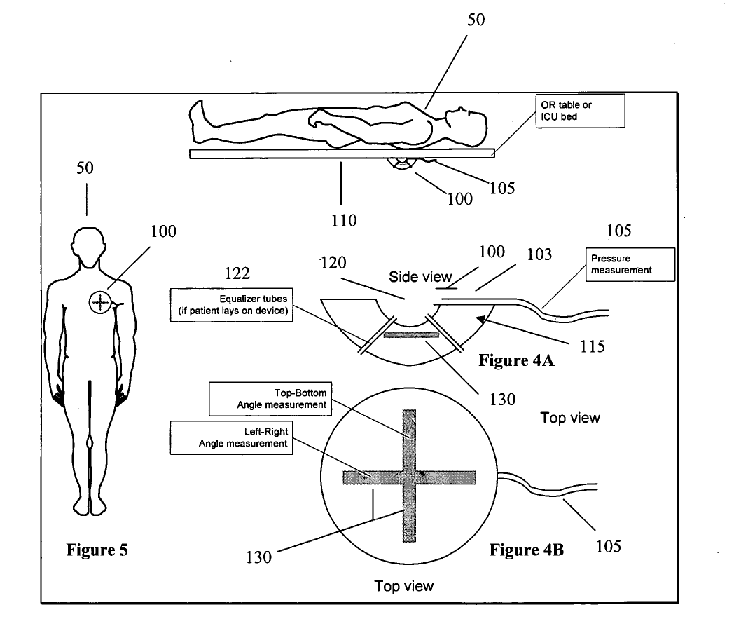

FIG. 4A shows a side view cross section of the reference pressure correction embodiment 100. The reference pressure correction 100 comprises a bracket or casing 115 that defines a chamber 120 which comprises channels 122 to allow equalization with atmospheric pressure. The distal end 103 of the line 105 is attached to or integrated with the casing 115. Attached to or integrated with the casing 115 is tilt sensor 130. The cross shape of the tilt sensor 130 in FIG. 4B is merely representative of the ability to sense tilt in two axes, and does not necessarily represent the shape and design of the inclinometer 130. The tilt sensor 130 may comprise two tilt sensing components such as electrolytic sensors configured as a dual axis arrangement. The reference pressure correction embodiment 100 may comprise an attachment means to readily attach it to a location under the table and strategically placed under the patient. For this purpose, the attachment means may be permanent or readily removable (such as by clips, button, hooks, hook and loop fabric, adhesives, etc.)

FIG. 5 shows a top view of a patient 50, wherein the patient 50 is transparent to show the positioning of the pressure correction unit 100 under the patient 50. Positioning the patient 50 and/or the pressure correction unit 100 such that the patient's heart is directly over the unit will maximize the accuracy of the pressure readout. The pressure correction unit 100 may be directly under the patient, i.e., contacting the patient's back. In such instance, the casing is preferably small but rigid. It should be rigid so that pressure from the patient does not crush the unit. Small and dimensioned so that pushing into the patient's back is minimized. Preferably, the pressure correction unit is located under the patient table. Accordingly, one embodiment of the subject invention pertains to an operating table, patient bed, or stretcher comprising a reference pressure sensor attached thereto.

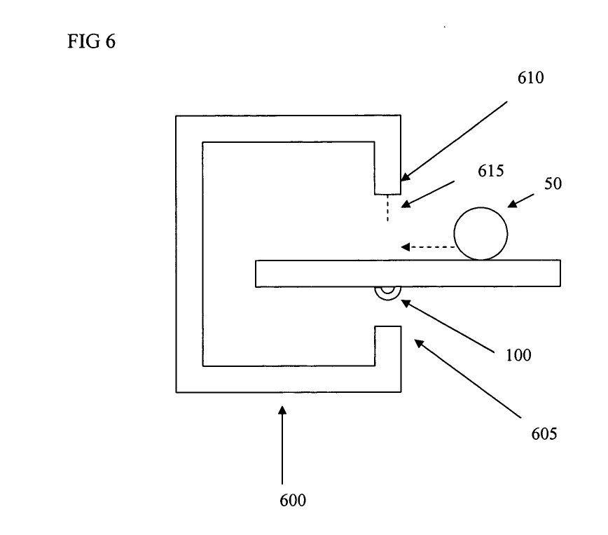

Furthermore, a device may be utilized to properly align the patient and the pressure correction unit. For example, a pre-measured bracket, such as a C-shaped or U-shaped bracket may be implemented. FIG. 6 shows a bracket embodiment 600 that may be used for this purpose. In use, the bottom end 605 of the bracket is positioned in line with the pressure correction unit 100 and the top 610 end is used as a guide to properly align the patient 50 such that the right atrium of the patient's heart (not shown) is directly above the reference sensor. Conversely, in embodiments where the reference sensor is removably attachable to the operating table, the top end is positioned above the patient's heart, and the bottom end is used to guide the placement of the pressure correction unit. The top end or bottom end may include a simple laser (hidden) or other light that emits a directional light 615 to assist the user in aligning either the patient or pressure correction unit. In an alternative embodiment, an operating table is equipped with an arm that can swing over the patient. The arm is of a size and dimension such that it guides proper placement of the patient on the operating table.

Those skilled in the art will recognize that conventional materials and manufacturing techniques may be utilized to make the subject devices. For example, the materials utilized in the components of the subject invention would be made of “plastics and polymers” as that these terms are broadly construed. Such materials may include, but are not limited to, polycarbonate, or similar plastics for housing sections and related components. One preferred polycarbonate material is LEXAN brand polycarbonate available from General Electric Company. Other specific preferred materials such as nylon or glass filled nylon (for strength) are also utilized. However, equivalent alternative materials will readily come to the mind of those skilled in the art.

Those skilled in the art will appreciate that pressure detectors and transducers used as the first catheter/transducer combination and/or one or more reference catheters/transducer combination could be any type known in the art which is capable of being zeroed to a reference. As used herein, the term “detector” broadly refers to a catheter, or similar means (e.g., tube, line, wire, or wireless) for detecting pressure changes and transferring the pressure change information to a transducer, that in turn, converts the actual pressure change information into an electrical signal. Preferably, such detectors and transducers will be characterized as having ‘high fidelity’ as that term is known in the art. This is preferred to avoid errors brought about by overshooting or undershooting the appropriate correction of the actual pressure of the patient. For example, the detectors/transducers should be able to detect changes in altitude with standard of error of no more than 1 cm. Preferably, the standard error is no more than 0.5 cm. Examples of detectors/transducers capable of being zeroed to a reference include, but are not limited to, conventional air filled or fluid filled single or multi-lumen catheters (with or without membranes or balloons on their distal tip) which are in communication with a pressure transducer. Examples of central venous catheters that may be used in accord with the teachings herein include those sold by Cook Critical Care (e.g. www.cookcriticalcare.com/discip/icc_pulm/2_{—}05/index.html). The pressure transducer coverts the pressure change into an electrical signal. Specialized multi-function catheters such as a Swanz-Ganz catheter; or catheters comprising a transducer at their tip such as a Millar Micro-Tip® Catheters (which are zeroed electrically based on the measurement obtained from the distal tip) may also be adapted for use in accordance with the devices and methods described herein. Furthermore, it is contemplated that catheter/transducer systems may be developed which are wireless, e.g., the transducer portion transmits the signal to a receiver. Such wireless transducers may be implemented in accord with the teachings herein.

A number of multi-system monitors on the market today utilize easily removable and configurable modules, each designed to process data regarding a specific measurement or patient vital sign. For example, the V24 and V26 systems (Phillips Medical Systems) comprise removable modules that receive and process single data measurements, such as EEG, SpO2, CVP, etc. Such a module is readily adapted to receive and process data from two or more pressure transducers. In one embodiment, the module could contain inputs to receive electrical information from the patient catheter/transducer, from the reference detector/transducer and from one or more tilt sensors. Preferably, the electrical inputs are combined together to form a single plug in socket arrangement with the module. The module contains the necessary hardware and software components to process the data from the two or more transducers by adjusting the patient's pressure value to take into account changes in tilt and elevation as detected by a reference pressure sensor and one or more tilt sensors. This is accomplished by a simple subtraction algorithm.

By way of example, to obtain a corrected pressure reading, a catheter is inserted into a patient and placed in the patient's vena cava at the right atrium (patient pressure sensor) and a detector is placed under the patient and aligned with the patient's right atrium. The patient catheter communicates with a first transducer and the reference detector communicates with a second transducer. After zeroing the patient catheter/transducer and the reference detector/transducer, the patients CVP reading is 10 mmHg. If the patient is raised 5 cm, without adjusting the corresponding first and second transducers, his CVP will increase from 10 mmHg (correct calculation) to 15 mmHg. Because the reference detector/transducer would detect an increase in 5 mmHg, a physician would know that the increase in CVP pressure is not actual, but only brought about by the patients raised elevation. In a basic embodiment, the individual values of the patient catheter/transducer and the reference detector/transducer are displayed separately, necessitating the physician to observe and process whether changes in pressure readings are actual or brought about by tilting or elevating/lowering the patient. In a preferred embodiment, the data from the patient catheter/transducer, the reference detector/transducer, and one or more tilt sensors are processed by basic hardware and software components, where the software comprises a simple subtraction algorithm. Thus, in the foregoing example, the reading from the reference detector/transducer and tilt sensor(s) is subtracted from the reading of the patient catheter/transducer and processed to display a single corrected reading. The hardware and software components may be comprised in a microcontroller packaged with or alongside a catheter/transducer system, or more preferably, are comprised in a module for conventional multi-system monitor systems as described above.

The teachings of the references cited throughout the specification are incorporated herein by this reference to the extent they are not inconsistent with the teachings herein. It should be understood that the examples and embodiments described herein are for illustrative purposes only and that various modifications or changes in light thereof will be suggested to persons skilled in the art and are to be included within the spirit and purview of this application and the scope of the appended claims.

1. A system for monitoring invasive pressures of a patient comprising at least one patient pressure catheter designed for placement in a patient; at least one reference pressure detector; a first transducer in communication with said at least one patient pressure catheter; a second transducer in communication with said at least one reference pressure detector; and at least one tilt sensor for sensing tilt of said patient.

2. The system of claim 1 wherein said first transducer is electrically connected to a pressure monitoring system.

3. The system of claim 1, wherein said at least one tilt sensor is affixed to or integrated into said at least one reference pressure detector.

4. The system of claim 1 wherein said at least one reference pressure detector is mounted to an operating table or an intensive care unit table.

5. The system of claim 1 wherein said at least one reference pressure detector comprises a casing to protect said pressure detector.

6. The system of claim 1, wherein said at least one patient pressure catheter is positioned in said patient at the opening of the right atrium

7. The system of claim 1 wherein said at least one reference pressure detector comprises a catheter comprising a distal end positioned at a location under the right atrium of said patient and a proximal end connected to said second transducer.

8. A system for monitoring invasive pressures of a patient comprising at least one patient pressure catheter designed for placement in a patient; at least one reference pressure detector; a first pressure transducer in communication with said at least one patient pressure catheter; and a second transducer in communication with said at least one reference detector; wherein said at least one reference pressure detector is mounted to an operating table or intensive care unit table.

9. The system of claim 8 wherein said at least one reference pressure detector comprises a chamber that is exposed to atmospheric conditions, wherein a first end of a tube connects to said chamber and a second end of said tube connects to said second pressure transducer.

10. The system of claim 8 wherein said at least reference pressure detector is mounted to the bottom of said operating table or intensive care unit table.

11. The system of claim 8 further comprising at least one tilt sensor mounted to said operating table or intensive care unit table.

12. The system of claim 11 wherein said at least one tilt sensor is affixed to or integrated with said at least one reference pressure detector.

13. A pressure correction unit for use in invasive pressure monitoring comprising a casing defining a chamber exposed to atmospheric conditions and at least one tilt sensor affixed to or integrated into said pressure correction unit.

14. The pressure correction unit of claim 13 wherein said tilt sensor is a dual axis accelerometer.

15. The pressure correction unit of claim 13 wherein said tilt sensor is electrically connected to a microcontroller, wherein said microcontroller is programmed to interpret tilt information from said tilt sensor and generate a value representing the change in elevation of a patient's right atrium.

16. The pressure correction unit of claim 13 attached to a catheter comprising a distal end and a proximal end; wherein said catheter is attached to said pressure correction unit at said distal end and connected to a pressure transducer at said proximal end.

17. A method for invasively monitoring pressures of a patient comprising positioning a catheter comprising a distal end and a proximal end in said patient such that said distal end is positioned at the patient's heart and said catheter is connected to a first pressure transducer at said proximal end; providing a reference pressure detector at an affixed location relative to said patient wherein said pressure detector is in communication with a second pressure transducer; changing the elevation of the patient; and modulating a pressure measurement of said patient obtained from said catheter to compensate for said change in elevation.

18. A method for invasively monitoring pressures of a patient comprising setting a reference pressure for a patient; tilting a patient; detecting the tilt of a said patient; calculating the change in altitude of a body part of said patient brought about by said tilting; and modulating a pressure measurement of said patient to compensate for said tilting.

19. An operating table comprising the reference pressure correction unit of claim 13 mounted thereto.
